(19) United States
(12) Patent Application Publication
(10) Pub. No.: US 2007/0078438 A1
(43) Pub. Date: Apr. 5, 2007

(54) Catheter assembly and sheath tear assistant tool

(75) Inventor: Yosuke Okada, Shizuoka (JP)

Correspondence Address: SNIDER & ASSOCIATES, P. O. BOX 27613, WASHINGTON, DC, 20038-7613, US

(73) Assignee: Okid Corporation, Shuchi-gun (JP)

(21) Appl. No.: 11/319,442

(22) Filed: Dec. 29, 2005

(30) Foreign Application Priority Data
Jul. 5, 2005 (JP) 195829/2005

(51) Int. Cl. A61M 25/00

(52) U.S. Cl. 604/523

(57) ABSTRACT

A catheter a part which is inserted into and indwelled in a blood vessel by the Seldinger method and a sheath for covering the catheter are included. The catheter is constituted of a double lumen portion in which a first lumen and a second lumen are integrally formed in parallel with each other, a first branch tube and a second branch tube extended to the one side of the double lumen portion and an extension tube connecting portion provided to the other side of the double lumen portion. The inner cavity of the first branch tube communicates with the first lumen and the inner cavity of the second branch tube communicates with the second lumen. The distal end of the first branch tube is bulged in the vicinity thereof so as to slidably contact with the inside of the distal end of the sheath.

### TECHNICAL FIELD

The invention relates to a catheter assembly which has good patency so that, even If the catheter is indwelled in a blood vessel for a long time, blood drain and feed ports are not occluded by thrombus or the blood vessel wall and the desired quantity of blood flow can be secured, and which is also easily inserted into and indwelled in the blood vessel. The invention also relates a sheath tear assistant tool.

### BACKGROUND ART

Hemodialysis is a medical treatment in which blood is taken out of from the body of a patient with renal failure, drug addiction or fulminant hapatitis and the like, removing toxins or water from the blood through an artificial kidney, and returning the treated blood into the patient's body. This medical treatment requires several hours for each and is performed several times per week. Therefore, stable blood outlet and inlet (blood drain and feed ports) are necessary over a long time. In order to make such blood drain and feed ports, a catheter for hemodialysis is often used.

The catheter for hemodialysis is used because of such necessity, and in particular, a double lumen catheter is widely used by which it is possible to take out of blood out of a patient's body (blood draining) and return the blood into the body (blood feeding) with one catheter and which is easily inserted into a blood vessel and can efficiently maintain the quantity of blood flow.

However, a large problem of the double lumen catheter is that an opening serving as blood drain and feed ports in a blood vessel is slowly occluded by thrombus or the blood vessel wall due to indwelling the catheter in the blood vessel over a long time, which can cause insufficient blood flow quantity.

In recent years, a method in which two single lumen catheters are inserted and indwelled side by side, has been performed for blood draining and blood feeding because long-time patency of the catheter can be obtained. As the difference from a conventional method for inserting and indwelling an integrated double lumen catheter, it is considered that, using this method, blood flows around the entire circumference of the vicinity of the distal end of each catheter, thus restraining occlusion of an opening.

Moreover, in case of this method, because each single lumen catheter independently moves on a blood flow, it is also reported that fibrin and other substances deposited in the vicinity of the distal end are decreased in quantity.

However, considering that even inserting and indwelling one catheter is troublesome, the method to insert and indwell two single lumen catheters side by side is more troublesome because two catheters must be individually inserted and indwelled. The method also causes difficulties in managing infection prevention because catheters are pierced into two portions of the body.

Therefore, as a catheter having only advantages of the above two types, in U.S. Pat. Ser. No. 6,001,079 and the like, a split tip catheter is suggested, which is a catheter with the structure in which one double lumen catheter at the proximal side is split into two catheters at the distal side.

However, this catheter has a disadvantage that it is very difficult to insert the catheter into a blood vessel. When inserting and indwelling this catheter, for example, a guide wire is previously placed in a blood vessel Then, the guide wire skewers the wall of a longer branch tube of the catheter, then being led to the distal end opening of a shorter tube of it, and under this state, the catheter is inserted into a blood vessel. However, when inserting the catheter by using this method, the insertion resistance of the catheter is very large. Therefore, it is impossible to insert the catheter unless the insertion portion is previously widely incised.

In another inserting method, a sheath is previously placed in a blood vessel and then a catheter is inserted through the inner cavity of the sheath. Specifically, a guide wire is previously placed in a blood vessel and a set of a sheath and dilator is inserted along the wire. Thereafter, the dilator is removed, leaving the sheath in the blood vessel. The distal end of the catheter is inserted into the sheath up to the predetermined position. Then, the sheath is removed. It is pulled back little by little so as not to pull the catheter back, and a portion of the sheath appearing outside the body is sequentially divided and this process is repeated until the entire sheath appears outside, so that the entire sheath is divided into two and removed.

A large problem of this method is the back flow of air from the sheath port. As the vena cava of the neck region in which a catheter for dialysis is preferably indwelled has a low blood pressure, it easily becomes a negative pressure due to breathing of a patient, draws air from the sheath port, which may cause complications such as air embolism. Therefore, immediately after extracting the dilator from the sheath, the operator has to close the sheath port. Moreover, when inserting the catheter, it is necessary to quickly insert long and short two branch tubes into the sheath in order to minimize lead-in of air. As another problem, two steps of placing the sheath and inserting the catheter are necessary, which causes a lot of trouble.

Moreover, as still another problem, because the wall sectional area is increased in split portion of the catheter compared to in the body portion of it, the inner cavity of the split portion becomes smaller, thus reducing the blood flow quantity.

[Patent Reference 1] U.S. Pat. No. 6,001,079

[Patent Reference 2] U.S. Pat. No. 5,947,953

Disclosure of the Invention

Problems to be Solved by the Invention

A problem to be solved is that inserting a split tip catheter into a blood vessel is difficult in terms of technique. Moreover, another problem to be solved is that the inner cavity becomes smaller in the split portion, reducing the blood flow quantity.

Means for Solving the Problems

The invention has the most important characteristic that, in order to make insertion of a split tip catheter into a blood vessel easy, the catheter is covered with a sheath except the distal end of a longer one of two-forked portions of the catheter and the vicinity of the portion contacting with the distal end of the sheath is bulged to minimize the gap between the portion and the sheath, forming the distal end of the catheter and the sheath like a truncated cone shape.

That is, a catheter assembly according to the invention comprises a catheter a part of which is inserted into and indwelled in a blood vessel by using the Seldinger method, and a sheath for covering the catheter, and in that the catheter comprises a double lumen portion in which a first lumen and a second lumen are integrally formed in parallel with each other, and a first branch tube and a second branch tube formed extending to one side of the double lumen portion, and an extension tube connecting portion provided to the other side of the double lumen portion, and the inner cavity of the first branch tube communicates with the first lumen, the inner cavity of the second branch tube communicates with the second lumen, the first branch tube is longer than the second branch tube, the sheath is substantially tubular shape and covers the first branch tube, the second branch tube, and the double lumen portion like a round bar so that it can be axially torn by hand, and moreover the vicinity of the distal end of the first branch tube is bulged so as to slidably contact with the inside of distal end of the sheath.

In this case, it is preferable that the vicinity of the distal end of the sheath is slightly diametrically reduced at the vicinity of the distal end of the first branch tube and slidably contacts with the first branch tube. Moreover, it is preferable that the distal end of the first branch tube is gradually diametrically reduced toward the side of the distal end. It is possible to use the first lumen as a flushing lumen, the second lumen as a aspiration lumen, the first branch tube as a blood flushing tube, and second branch tube as a blood aspiration tube.

All or a part of the first branch tube and second branch tube may be made of a hydrophilic swelling material over the overall circumference or a part of circumference. Moreover, a part of the facing side of the first branch tube and/or the second branch tube at the facing side may be made of a hydrophilic swelling material.

Furthermore, a sheath tear assistant tool according to the invention is characterized by comprising a control part for slidably controlling the spread of the sheath from the side at the portion of one side of the double lumen portion when tearing the sheath, and a stop part for stopping slipping out of the double lumen portion to the other side of the double lumen portion by holding the other side portion of the double lumen when tearing the sheath.

In this case, the stop part may be a stop part to be inserted into the joint portion between the first extension tube and the second extension tube and a gripper for gripping any other side portion of the double lumen.

Moreover, another sheath tear assistant tool according to the invention comprises an annular fixing part, a pair of wing parts integrally formed with the fixing part at the both sides of the fixing part, and a pair of picking parts connected to each end of a torn sheath, and a hole for passing the torn sheath is opened in the pair of wing parts and the diameter of the hole is smaller than the outside diameter of the picking part.

Advantageous Effect of the Invention

In the case of a catheter assembly of the invention, two branch tubes of a split tip catheter are integrally covered with a seath, forming one round bar shape with truncated cone shaped distal end. Therefore, there is an advantage that a split tip catheter whose insertion is normally difficult can be easily inserted into and indwelled in a blood vessel.

In the case of a catheter assembly of the invention, two branch tubes at the distal side of a split tip catheter are integrally covered with a sheath so that the catheter and the sheath can be simultaneously inserted. Therefore, there is an advantage that air is not back-flown into a blood vessel from a sheath port.

Moreover, in the case of a catheter assembly of the invention, the vicinity of the distal end of the first branch tube is bulged so as to slidably contact with the inside at the distal end of the sheath. Therefore, there is an advantage that the catheter can be smoothly inserted and indwelled into and in a blood vessel.

Furthermore, in the case of a catheter assembly of the invention, if the vicinity of the distal end of the sheath is slightly diametrically reduced at the vicinity of the distal end of the first branch tube and slidably contacts with the first branch tube, there is an advantage that the catheter can be smoothly inserted and indwelled into and in a blood vessel.

Furthermore, in the case of a catheter assembly of the invention, if the vicinity of the distal end of the first branch tube is gradually diametrically reduced toward the distal end side and forms a truncated cone shape, there is an advantage that the catheter can be smoothly inserted and indwelled into and in a blood vessel.

Furthermore, in the case of a catheter assembly of the invention, if all or a part of the first branch tube and second branch tube may be made of a hydrophilic swelling material over the overall circumference or a part of circumference, these branch tubes are swelled by the water in blood and their inner cavities are expanded. Therefore, there is an advantage that it is possible to maintain the desired quantity of blood flow.

Furthermore, in the case of a catheter assembly according to the invention, if all or a part of the first branch tube and all or a part of the second branch tube are made of with hydrophilic swelling material over the circumference, these branch tubes swell by the water in blood and their wall thickness increases. Therefore, there is an advantage that the first branch tube and the second branch tube are prevented from being bent.

Furthermore, in the case of a catheter assembly, if all or a part of the first branch tube and all or a part of the second branch tube are or is made of a hydrophilic swelling material over only a part of the circumference, it is possible to suppress the reduction of the pulling strength of the branch tubes, which occurs in branch tubes made of hydrophilic swelling material over the circumference, and it is also possible to suppress the reduction of the fusion-bonding force with the double lumen portion. Moreover, because axial. expansion of hydrophilic swelling material is suppressed, radial expansion of it is increased, so that cross sectional area of the inner cavity of the branch tube is increased.

Furthermore, in the case of a catheter assembly according to the invention, if the side of the first branch tube and/or the side of the second branch tube which are facing each other are or is partially made of hydrohilic swelling material, the portion made of hydrophilic swelling material locally swells in inserting the catheter into a blood vessel. Consequently, the first branch tube and/or the second branch tube are or is bent outward, preventing them from approaching each other. Therefore, there is an advantage that deposition of fibrin and the like on blood drain and blood feed ports is prevented.

Furthermore, a sheath tear assistant tool according to the invention has an advantage that a sheath can be tom and removed by a person without extracting an inserted and indwelled catheter.

### BEST MODE FOR CARRYING OUT THE INVENTION

A purpose to make it easy to insert a split tip catheter whose insertion and indwelling are difficult was achieved with a simple configured catheter assembly without losing the patency of a catheter.

FIG. 1 shows a perspective view of a catheter assembly according to an embodiment of the invention and FIG. 2 shows a longitudinal sectional view of the distal end of the catheter in FIG. 1. As shown in these drawings, a catheter assembly 10 consists of a split tip catheter 12 and a sheath 14 for covering the split tip catheter 12.

The distal end of the split tip catheter 12 slightly protrudes from the distal end of the sheath 14. Moreover, a stylet 16 is inserted into the split tip catheter 12 and the distal end of the stylet 16 protrudes from the distal end of the split tip catheter 12.

FIG. 3 shows a perspective view of a split tip catheter in the catheter assembly in FIG. 1, FIG. 4 shows a sectional view taken along the line A-A in FIG. 3, FIG. 5 shows a sectional view taken along the line B-B in FIG. 3, FIG. 6 shows a sectional view taken along the line C-C, and FIG. 7 shows a longitudinal sectional view of the distal end of the split tip catheter in FIG. 3.

As shown in FIG. 3, the split tip catheter 12 consists of a double lumen portion 18, a branch tube 20 extended from one side of the double lumen portion 18, and an extension tube connecting portion 36 set to the other side of the double lumen portion 18 and an extension tube 22 is connected to the extension tube connecting portion 36.

The double lumen portion 18 consists of a cylindrical body and inside it a first lumen 24 and a second lumen 26 which consist of cavities are integrally formed in parallel with each other as shown in FIG. 4.

The branch tube 20 is constituted of a first branch tube 28 and a second branch tube 30. As shown in FIG. 5, the first branch tube 28 consists of the cylindrical body whose cross section is substantially semicircular and the hole of the inside of the first branch tube 28 is connected with the first lumen 24. As shown in FIG. 5, the second branch tube 30 also consists of the cylindrical body whose cross section is substantially semicircular and the hole in the second branch tube 30 is connected with the second lumen 26.

The first branch tube 28 is longer than the second branch tube 30 and at the distal end of the first branch tube 28, the tube wall is partially thickened and thus the tube becomes bulged as a whole. Moreover, a gentle outline is continuously formed from the distal end of the first branch tube 28 up to the distal end of the sheath 14.

The extension tube 22 connects the catheter 12 to a dialysis system, and is constituted of a first extension tube 32 and a second extension tube 34. The first extension tube 32 consists of the cylindrical body whose cross section is substantially circular and the hole in the first extension tube 32 is connected with the first lumen 24. The second extension tube 34 also consists of the cylindrical body whose cross section is substantially semicircular and the hole in the second extension tube 34 is also connected with the second lumen 26 via the extension tube connecting portion 36.

The first branch tube 28, first lumen 24, and first extension tube 32 are used for blood feeding and the second branch tube 30, second lumen 26, and second extension tube 34 are used for blood draining.

As the material of the split tip catheter 12, polyurethane, nylon elastomer, and silicone rubber and the like are used. Particularly, as polyurethane has appropriate flexibility and strength, a sufficient quantity of blood flow can be secured by designing to make the inner cavity larger, the inner cavity be hardly closed when the catheter is bent and a blood vessel wall be hardly damaged. Therefore, the polyurethane is a preferable material.

When the branch tube 20 can be firmly adhered or fused with the double lumen portion 18, it is unnecessary to use the same material and same hardness. It is also possible to apply the same material as the double lumen portion 18 to the extension tube 22. If necessary, a soft material is selected.

The sheath 14 consists of a thin plastic cylindrical body. As a material of the sheath 14, it is preferable to use a material having strength and rigidity such as polyethylene, polypropylene, nylon elastomer, fluorine resin and the like which are not crushed even if the sheath is formed thin.

The dividing property of the sheath 14 can be provided by adding orientation when forming a tube or in a succeeding process so that the sheath can be torn from any direction in the surrounding area or arranging a plurality of weak lines along the longitudinal direction of the tube by using a single material or a blend of plurality materials.

Then, a method for inserting and indwelling a catheter into and in a blood vessel by using the catheter assembly according to the Seldinger method is described below.

First, an introducing needle for introducing a guide wire is pierced into a blood vessel from the outside of skin, the guide ware is inserted into the blood vessel via the introducing needle, and then the introducing needle is removed.

Then, the guide wire is inserted into the inside from the distal end of the stylet 16 inserted into the catheter assembly 10, the guide wire is taken out from the rear end of the stylet 16, and the catheter assembly 10 is approached to a inserting portion along the guide wire. Then, the distal end of the sheath 14 is pushed in the skin along the guide wire, and inserted into a blood vessel, and then the guide wire is removed.

Then, the sheath 14 is removed. When removing the sheath 14, the sheath tear assistant tool 38 is used as shown in FIG. 8. The sheath tear assistant tool 38 comprises a control part 40 for slidably controlling the spread of the sheath from the side when tearing the sheath 14, and a stop part 42 to be inserted into the joint portion between the first extension tube 32 and the second extension tube 34. The control part 40 and stop part 42 are integrally formed by a connection member 44.

It is preferable to use a hard and slippery material such as high-density polyethylene or polypropylene which can be easily injection-molded for the sheath tear assistant tool 38.

By pulling the both ends of the sheath 14 to right and left, a back sheath having inserted into a blood vessel is also extracted and torn at the point of control part 40. If pulling the sheath 14 without the sheath tear assistant tool 38, the catheter 12 is extracted together with the sheath 14. However, if pulling the sheath 14 to right and left, the sheath 14 presses the control part 40 in the direction of pressing it back to the body side and the stop part 42 integrated with the control part 40 presses the catheter 12 in the direction of pressing it back to the body side. Therefore, the catheter 12 is not extracted. Moreover, to tear a sheath, it is allowed to use the sheath tear assistant tool shown in FIG. 9. The sheath tear assistant tool 38 is constituted of a ring fixing portion fitted to the extension tube connecting portion 36, a pair of vane portions 48 and 48 formed integrally with the fixing portion 46 at the both sides of the fixing portion 46, and a pair of picking portions 50 and 50 connected to ends of the torn sheath 14. A hole for passing the torn sheath 14 is opened on the vane portions 48 and 48 and the diameter of the hole is smaller than the outside diameter of the picking portion 50. The fixing portion 46 is completely or rotatably fixed to the extension tube connection portion 36.

The back sheath 14 inserted into a blood vessel is also extracted by pulling the picking parts 50 and 50 to right and left and torn between wing parts 48 and 48 or in the region of the front of the fixing part 46. If pulling the sheath 14 without the sheath tear assistant tool 38, the catheter 12 is also extracted together with the sheath 14. However, if pulling the sheath 14 to right and left, the sheath 14 presses the sheath tear assistant tool 38 together with the catheter in the direction of pressing them to the body side. Therefore, the catheter 12 is not extracted.

The sheath tear assistant tool 38 can be used as a member for fixing a catheter to skin after tearing and removing the sheath 14.

If necessary, the stylet 16 to be applied is slidably mounted to the inner cavity of the split tip catheter 12, which is used to easily insert the catheter 12 into the body by increasing the rigidity of the catheter 12 or reduce the insertion resistance of the catheter 12 by filling the gap between the opening at the distal end of the branch tube on the blood feed side and a guide wire to be applied. It is preferable to use a strong and hard material such as high-density polyethylene, polypropylene, or nylon and the like for the stylet 16.

In order to simply increase the rigidity, it is possible to use solid and hollow stylets. However, in order to fill the gap of the latter, a hollow stylet through which a guide wire actively passes must be used.

It is preferable that the wall thicknesses of the first branch tube 28 and the second branch tube 30 are equal to or thicker than the wall thickness of the double lumen portion 18 in order to prevent them from being bend. Consequently, the inner cavities of the both branch tubes 28 and 30 become narrower than the inner cavity of the double lumen portion 18, reducing the blood flow quantity. Therefore, as shown in FIGS. 10 to 12, all or a part of the first branch tube 28 and all or a part of the second branch tube 30 may be made of a hydrophilic swelling material over the circumference.

If all or a part of the first branch tube 28 and all or a part of the second branch tube 30 are or is made of a hydrophilic swelling material over the circumference, in inserting the catheter 12 into blood, the first branch tube 28 and the second branch tube 30 absorb water in blood and swell, so that, as shown in FIG. 11(b), the inside diameter is expanded from h1 to h2 (h1<h2), making the circulation of blood better.

Moreover, if making the first branch tube 28 and the second branch tube 30 of a hydrophilic swelling material but making a part of them of a hydrophobic material as shown in FIG. 13(a), the reduction of the pulling strength of both the first branch tube 28 and the second branch tube 30 is suppressed and the fusion-bonding with the double lumen portion also can be further secured.

Furthermore, when the hydrophilic swelling material absorbs water in blood and swells, the first branch tube 28 and the second branch tube 30 swell from the semicircular shape approaching to a circular shape, further expanding the cross sectional areas of the inner cavities, so that the circulation of blood becomes better as shown in FIG. 13(b).

Furthermore, according to the invention, because the first branch tube 28 and the second branch tube 30 are housed in the sheath 14, they approach each other inside the narrow sheath while being kept there for a long time and even after they are released from the sheath in the blood vessel, they tend to be close to each other. It is said that if the first branch tube 28 and the second branch tube 30 are distant from each other to a certain extent in a blood vessel, blood flows around the tubes, preventing the occlusion of an opening and the deposition of fibrin and the like. However, if they are close to each other, such advantages are not exhibited. Therefore, as shown in FIGS. 14(a) and 15, the side of the second branch tube 30 which faces the first branch tube 28 may be partially made of a hydrophilic swelling material.

If making the side of the second branch tube 30 facing the first branch tube 28 partially of the hydrophilic swelling material, in inserting the catheter 12 into blood, the portion of the hydrophilic swelling material absorbs water in blood and swells, and as shown in FIG. 14(b), the second branch tube 30 is bent and moved away from the first branch tube 28, so that fibrin and the like in blood is hardly deposited, increasing the patency.

### MANUFACTURED EXAMPLE 1

A catheter 12 was made of aromatic isocyanate polyurethane containing 20% of barium sulfate with JIS hardness of 90 degree and consisted of 12Fr×10 cm double lumen portion 18, the first branch tube 28 in the length of 12 cm, and the second branch tube 30 in the length of 7 cm. The branch tube 28 and 30 and the double lumen portion 18 was fusion-bonded each other by passing a cored bar through each inner cavity of the branch and the double lumen portion, covering them with a silicone heat-shrinkable tube with pressing them each other, and heating to contract the tube.

The first branch tube 28 was formed so that the outer shape of it was 12 Fr circle between 15 and 25 mm from the distal end of the tube, from where to the distal end the diameter of the tube was decreased, forming a cone shape, and from where towards the proximal side the diameter was slowly decreased over 10 mm to restore the tube to the original shape. A nylon hollow stylet was mounted over all length of a flushing lumen with the distal end of the stylet protruding by 10 mm from the distal end of the first branch tube 28. The catheter was covered with the splittable sheath 14 made of 13.5-Fr fluorine resin. The splittable sheath 14 was mounted so that the distal end of the sheath was located at 20 mm from the distal end of the first branch tube 28.

### MANUFACTURED EXAMPLE 2

A catheter 12 was formed by making double lumen portion 18 in 12Fr×10 cm of aromatic isocyanate polyurethane containing 20% of barium sulfate with Shore D hardness of 65 degree and by making the first branch tube 28 in the length of 10 cm and the second branch tube 30 in the length of 6 cm of the same material having Shore A hardness of 85 degree.

The both branch tubes 28 and 30 and the double lumen portion 18 were fusion-bonded each other by passing a cored bar through each inner cavity, covering them with a silicone heat-shrinkable tube with pressing them each other, and heating to contract them. A hollow stylet made of PP was mounted over all length of a flushing lumen and the distal end of the stylet protruded by 10 mm from the distal end of the first branch tube 28. And, a solid stylet made of PP was mounted to a aspiration lumen so that the distal end of the stylet was located at 5 mm proximally from the distal of the second branch tube 30.

The catheter was covered with the weak-line-contained splittable sheath 14 made of 13.5-Fr PP. The weak-line-contained splittable sheath 14 was mounted so that the distal end of the sheath was located at 20 mm from the distal end of the first branch tube 28. The sheath tear assistant tool 38 made of PP was mounted at the extension tube connecting portion 36.

### BRIEF DESCRIPTION OF THE DRAWINGS

FIG. 1 is a perspective view of a catheter assembly according to one embodiment of the invention.

FIG. 2 is a longitudinal cross-sectional view of the vicinity of the distal end of the catheter assembly in FIG. 1.

FIG. 3 is a perspective view of a split tip catheter in the catheter assembly in FIG. 1.

FIG. 4 is an A-A cross sectional view of FIG. 3.

FIG. 5 is a B-B cross sectional view of FIG. 3.

FIG. 6 is a C-C cross sectional view of FIG. 3.

FIG. 7 is a longitudinal cross-sectional view of a substantial part of the split tip catheter in FIG. 3.

FIG. 8 is an illustration showing a state in which a sheath tear assistant tool is mounted to a catheter assembly.

FIG. 9 is an illustration showing a state in which another sheath tear assistant tool is mounted to a catheter assembly.

FIG. 10 is a longitudinal cross-sectional view of the vicinity of the distal end of a split tip catheter of another embodiment of the invention.

FIGS. 11(a) and 11(b) are D-D cross sectional views of FIG. 10.

FIG. 12 is a cross-sectional view taken along the line E-E in FIG. 10.

FIGS. 13(a) and 13(b) are cross-sectional views of a branch tube of a tip catheter of another embodiment of the invention.

FIGS. 14(a) and 14(b) are longitudinal cross-sectional views of the vicnity of the distal end of a split tip catheter of another embodiment of the invention.

FIG. 15 is an F-F cross sectional view of FIG. 14(a).

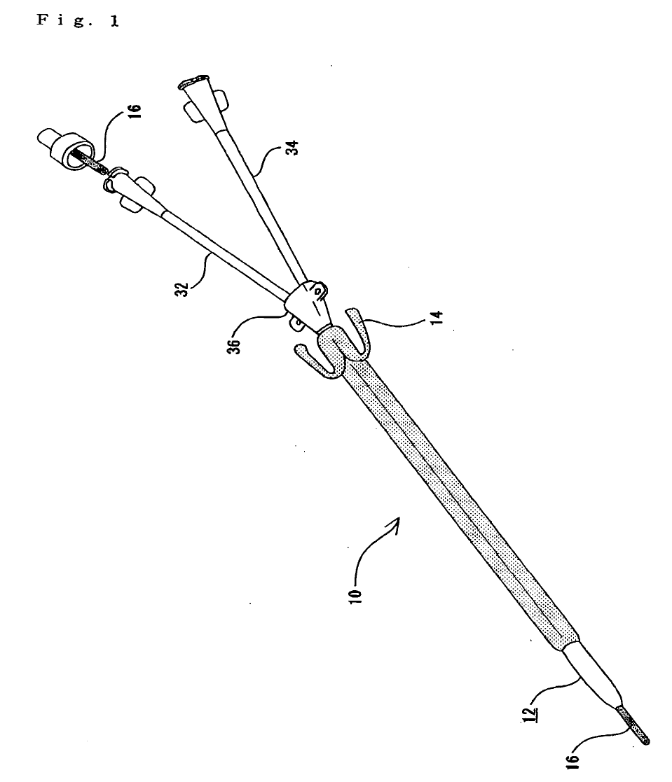

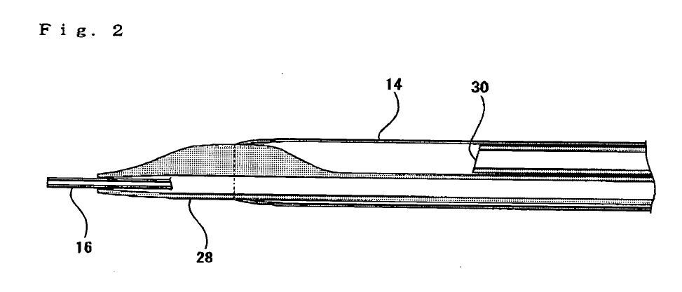

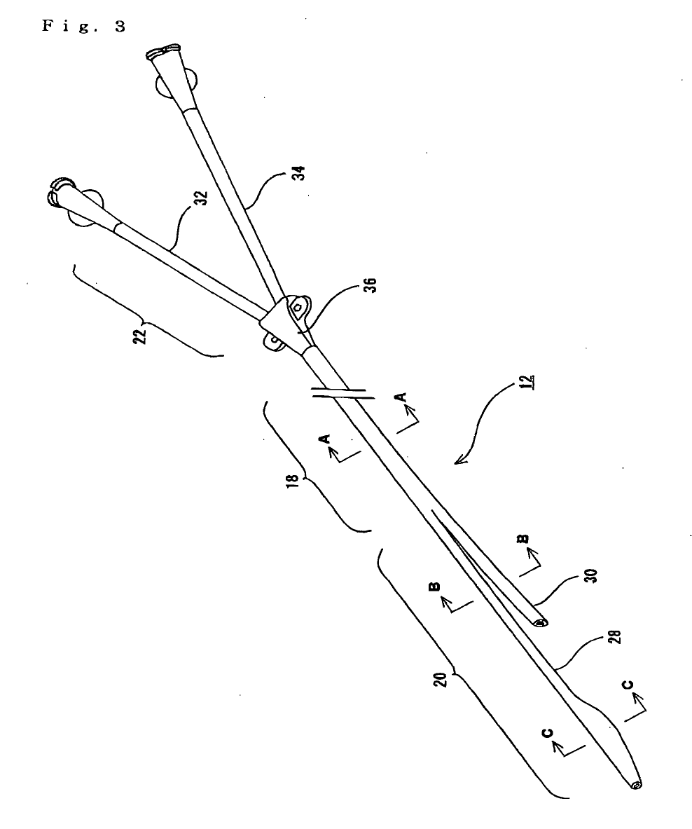

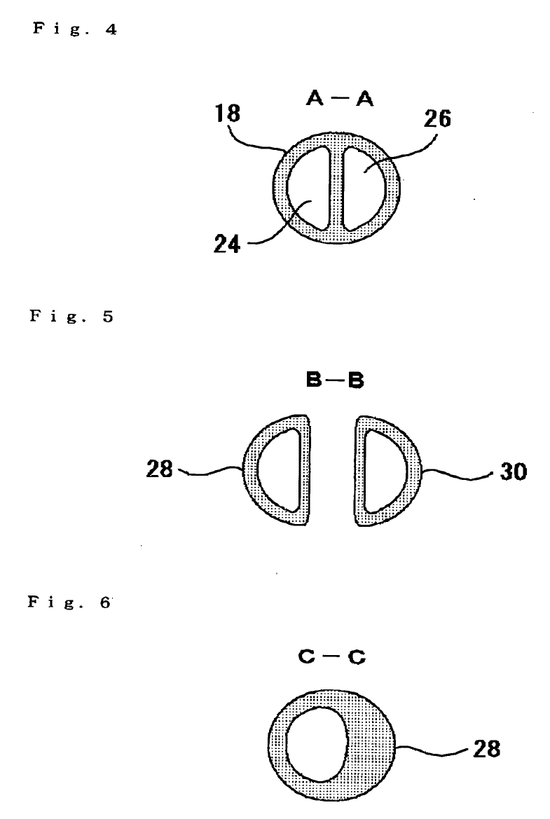

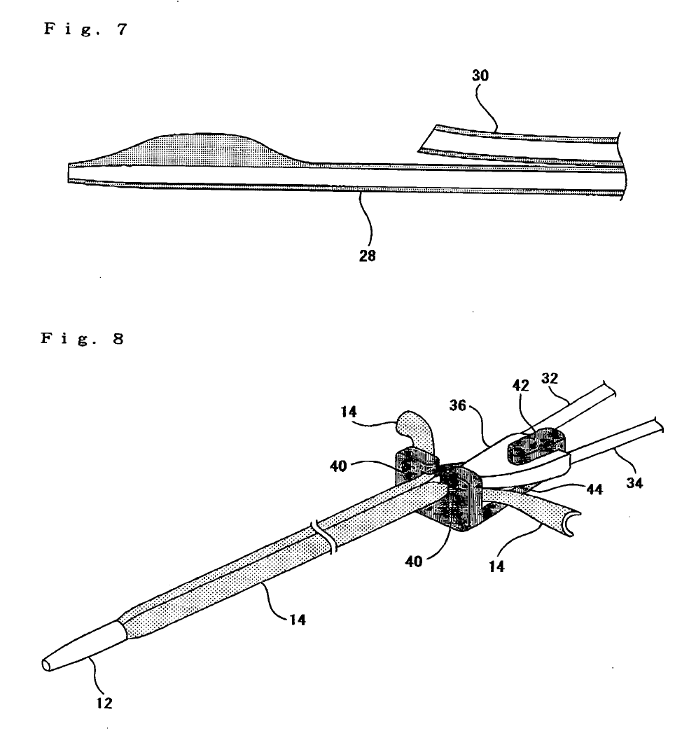

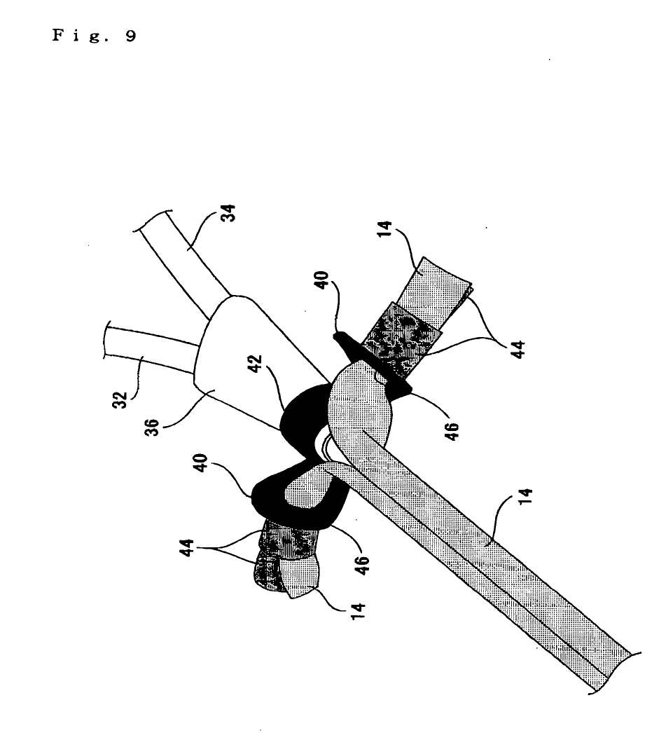

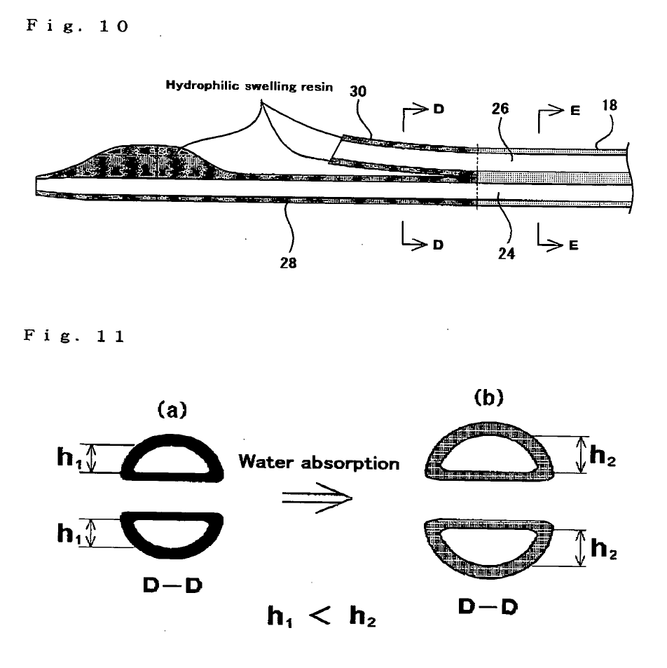

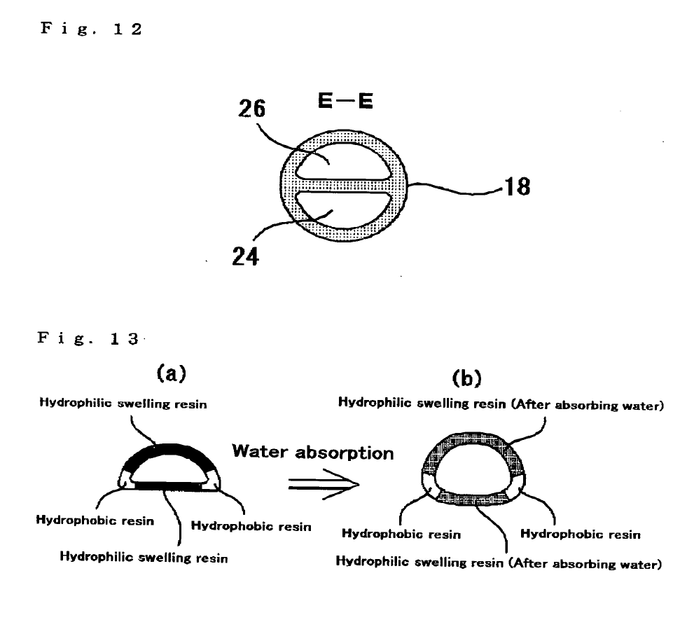

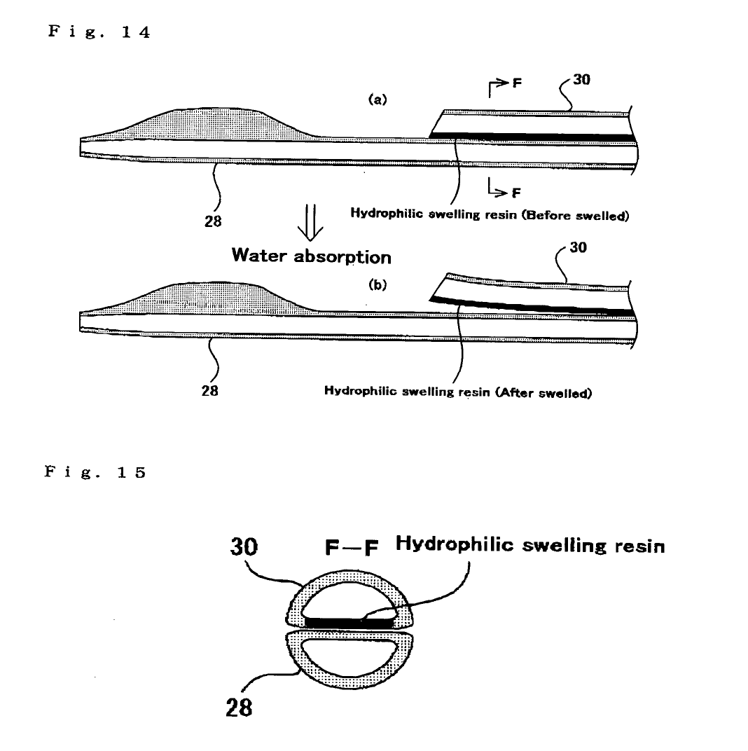

### EXPLANATION OF NUMERALS

10 catheter assembly
12 split tip catheter
14 seath
16 stylet
18 double lumen portion
20 branch tube
22 extension tube
24 first lumen
26 second lumen
28 first branch tube
30 second branch tube
32 first extension tube
34 second extension tube
38 seath assistant tool
40 control part
42 stop part
44 connecting member
46 fixing part
48 wing part
50 picking part
52 hole

1. A catheter assembly comprising:
a catheter a part of which is inserted into and indwelled in a blood vessel by using the Seldinger method; and
a sheath for covering the catheter, the catheter having:
a double lumen portion in which a first lumen and a second lumen are integrally formed in parallel with each other;
a first branch tube and a second branch tube formed extending to one side of the double lumen portion; and
an extension tube connecting portion provided to the other side of the double lumen portion,
the inner cavity of the first branch tube communicating with the first lumen, the inner cavity of the second branch tube communicating with the second lumen, the first branch tube being longer than the second branch tube,
the sheath being substantially tubular shape and covering the first branch tube, the second branch tube, and the double lumen portion like a round bar so that it can be axially torn by hand,
moreover the distal end of the first branch tube being bulged in the vicinity thereof so as to slidably contact with the inside of distal end of the sheath.

2. The catheter assembly of claim 1, wherein the vicinity of the distal end of the sheath is diametrically reduced at the vicinity of the distal end of the first branch tube and contacts with the first branch tube.

3. The catheter assembly of claim 1, wherein the vicinity of the distal end of the first branch tube is gradually diametrically reduced toward the distal end side.

4. The catheter assembly of claim 1, wherein the distal end of the first branch tube is gradually diametrically reduced toward the side of the double lumen portion.

5. The catheter assembly of claim 1, wherein the first lumen is a flushing lumen, the second lumen is an aspiration lumen, and the first branch tube is a blood flushing branch tube, and the second branch tube is a blood aspiration branch tube.

6. The catheter assembly of claim 1, wherein all or a part of the first branch tube and second branch tube is made of a hydrophilic swelling material over the overall circumference.

7. The catheter assembly of claim 1, wherein all or a part of the first branch tube and second branch tube is made of a hydrophilic swelling material at a part of circumference.

8. The catheter assembly of claim 1, wherein a part of the facing side of the first branch tube and/or the second branch tube at the facing side is made of a hydrophilic swelling material.

9. A sheath tear assistant tool comprising:
a control part for slidably controlling the spread of the sheath from the side at the portion of one side of the double lumen portion when tearing the sheath, and
a stop part for stopping slipping out of the double lumen portion to the other side of the double lumen portion by holding the other side portion of the double lumen when tearing the sheath.

10. The sheath tear assistant tool of claim 9, wherein the stop part is constituted of a stop part to be inserted into the joint portion between the first extension tube and the second extension tube.

11. The sheath tear assistant tool of claim 9, wherein the stop portion is constituted of a holder for holding any other side portion of the double lumen.

12. A sheath tear assistant tool comprising:
an annular fixing part,
a pair of wing parts integrally formed with the fixing part at the both sides of the fixing part, and
a pair of picking parts connected to each end of a torn sheath,
a hole for passing the torn sheath being opened in the pair of wing parts and the diameter of the hole being smaller than the outside diameter of the picking part.

13. The catheter assembly of claim 2, wherein the vicinity of the distal end of the first branch tube is gradually diametrically reduced toward the distal end side.

14. The catheter assembly of claim 2, wherein the distal end of the first branch tube is gradually diametrically reduced toward the side of the double lumen portion.

15. The catheter assembly of claim 3, wherein the distal end of the first branch tube is gradually diametrically reduced toward the side of the double lumen portion.

16. The catheter assembly of claim 2, wherein the first lumen is a flushing lumen, the second lumen is an aspiration lumen, and the first branch tube is a blood flushing branch tube, and the second branch tube is a blood aspiration branch tube.

17. The catheter assembly of claim 3, wherein the first lumen is a flushing lumen, the second lumen is an aspiration lumen, and the first branch tube is a blood flushing branch tube, and the second branch tube is a blood aspiration branch tube.

18. The catheter assembly of claim 4, wherein the first lumen is a flushing lumen, the second lumen is an aspiration lumen, and the first branch tube is a blood flushing branch tube, and the second branch tube is a blood aspiration branch tube.

19. The catheter assembly of claim 5, wherein all or a part of the first branch tube and second branch tube is made of a hydrophilic swelling material over the overall circumference.

20. The catheter assembly of claim 5, wherein all or a part of the first branch tube and second branch tube is made of a hydrophilic swelling material as a part of circumference.
